(19) United States
(12) Patent Application Publication
(10) Pub. No.: US 2022/0175357 A1
(43) Pub. Date: Jun. 9, 2022

(54) SYSTEM AND METHOD FOR SELECTIVELY SEALING SMALL VESSELS

(71) Applicant: Covidien LP, Mansfield, MA (US)

(72) Inventors: Weijiang Ding, Shanghai (CN); Yuanxun Li, Shanghai (CN); Ming Cao, Shanghai (CN)

(21) Appl. No.: 17/603,640

(22) PCT Filed: May 13, 2019
(86) PCT No.: PCT/CN2019/086563

§ 371 Date: Oct. 14, 2021

(51) Int. Cl. A61B 17/00; A61N 7/02

(52) U.S. Cl. CPC A61B 17/0057; A61N 7/02; A61N 2007/0078; A61B 2017/00778; A61B 2017/00646

(57) ABSTRACT

A high intensity focused ultrasound (HIFU) system (100,400) for selectively sealing a vessel network (360) in a liver includes a generator (110,410,420) configured to generate and supply electric power, and an acoustic assembly (200,130,150) configured to receive the supplied electric power. The acoustic assembly (200,130,150) includes a first transducer (310,430) configured to generate vibrations having a first frequency, and a second transducer (320,440) configured to generate vibrations having a second frequency. When a first focal point of the generated vibrations having the first frequency and a second focal point of the generated vibrations having the second frequency are aligned within a focused region (350), a group of vessels (360) at the focused region (350) are selectively sealed.

### FIELD

The present disclosure relates to a system and method for selectively sealing a network of small vessels in a liver. More particularly, the present disclosure relates to a system and method for selectively sealing the network of small vessels in the liver using high intensity focused ultrasound (HIFU).

### BACKGROUND

Generally, water jet and cavitron ultrasonic surgical aspirator (CUSA) have been used in liver resection. Water jet is like an intelligent knife, which automatically separates resistant duct and vessel structures of the liver from parenchyma. The water jet hits the liver at a desired line of transection and washes away the parenchyma, leaving the intra-hepatic ducts and vessel undamaged. Then the vascular and bile structures can be ligated, and the transection plane can be coagulated. Even though the water jet can offer excellent visualization and is effective in cirrhotic liver, this technique has difficulty coagulating or realizing hemostasis and difficulty achieving a reduction of intra-operative blood loss and a slow operating time if compared with traditional techniques. Further, splashes from the water jets can cause contamination of an operating room, cancerous seeding of healthy abdominal organs, and infection of the operators by hepatic viruses.

CUSA is a surgical system in which a pencil-grip surgical hand piece contains a transducer, which oscillates longitudinally at 23 kHz. The vibrating tip of the instrument causes explosion of cells having high water content but spares blood and bile vessel because their walls are prevalently composed of connective cells including poor water content but rich of intracellular bonds. CUSA is further equipped with a saline solution irrigation system that removes fragmented bits of tissue and permits excellent visualization. However, there is continuing interest in improving technologies for selectively sealing small vessel network in liver surgeries.

### SUMMARY

The present disclosure is directed to a system and method for selectively sealing a network of vessels in a liver. More particularly, the system and method are directed to performing liver resection procedures by selectively exposing the network of blood vessels in the liver using high intensity focused ultrasound and sealing and resecting the exposed vessel network using other surgical tools such as a surgical knife once the vessel network is exposed.

Aspects of the present disclosure are described in detail with reference to the figures wherein like reference numerals identify similar or identical elements. According to one aspect of the present disclosure, high intensity focused ultrasound (HIFU) system for selectively sealing vessel network in a liver includes a generator configured to generate and supply electric power, and an acoustic assembly configured to receive the supplied electric power. The acoustic assembly includes a first transducer configured to generate vibrations having a first frequency, and a second transducer configured to generate vibrations having a second frequency. When a first focal point of the generated vibrations having the first frequency and a second focal point of the generated vibrations having the second frequency are aligned within a focused region, a group of vessels at the focused region are selectively sealed.

In an aspect, the first transducer has a first curvature and the second transducer has a second curvature. The first curvature is equal to the second curvature.

In another aspect, the group of vessels is not sealed when the first focal point and the second focal point are not aligned within the focused region.

In still another aspect, the second frequency is a harmonic frequency of the first frequency.

In still another aspect, the group of vessels has a diameter less than or equal to 0.2 millimeters.

In still another aspect, vessels, which have a diameter larger than 0.2 millimeters, are not sealed even when the first focal point and the second focal point are aligned within the focused region.

In still another aspect, the HIFU system further includes a first lens coupled with the first transducer and a second lens coupled with the second transducer.

In still another aspect, the first lens focuses the generated vibrations having the first frequency in the first focal point and the second lens focuses the generated vibrations having the second frequency in the second focal point.

In still another aspect, the HIFU system further includes a phase controller configured to adjust relative phases of elements of the first transducer and of the second transducer.

In yet another aspect, the phase controller focuses the generated vibrations having the first frequency at the first focal point and focuses the generated vibrations having the second frequency at the second focal point.

In another embodiment, a method for selectively sealing a vessel network with a high intensity focused ultrasound (HIFU) system including a first transducer and a second transducer includes providing electric power to the first transducer and the second transducer, generating vibrations having a first frequency by the first transducer, generating vibrations having a second frequency by the second transducer, aligning a first focal point of the generated vibrations having the first frequency and a second focal point of the generated vibrations having the second frequency are aligned within a focused volume, selectively sealing a group of vessels located at a focused region, when a first focal point of the generated vibrations having the first frequency and a second focal point of the generated vibrations having the second frequency are aligned within the focused volume.

In an aspect, the vibration having the first frequency are generated by the first transducer having a first curvature, and the vibration having the second frequency are generated by the second transducer having a second curvature.

In another aspect, the electric power provided to the first transducer is equal to the electric power provided to the second transducer. Or the electric power provided to the first transducer is equal to the electric power provided to the second transducer.

In still another aspect, the group of vessels is not sealed when the first focal point and the second focal point are not aligned within the focused volume.

In still another aspect, the second frequency is a harmonic frequency of the first frequency.

In still another aspect, the group of vessels is sealed when diameters of the group of vessels are less than or equal to 0.2 millimeters.

In still another aspect, vessels, which have a diameter larger than 0.2 millimeters, are not sealed even when the first focal point and the second focal point are aligned within the focused volume.

In yet another embodiment, a non-transitory computer readable medium including computer-executable instructions that, when executed by a computer, cause the computer to perform a method for selectively sealing a vessel network with a high intensity focused ultrasound (HIFU) system including a first transducer and a second transducer. The method includes providing electric power to the first transducer and the second transducer, generating vibrations having a first frequency by the first transducer, generating vibrations having a second frequency by the second transducer, aligning a first focal point of the generated vibrations having the first frequency and a second focal point of the generated vibrations having the second frequency are aligned within a focused volume, selectively sealing a group of vessels located at a focused region, when a first focal point of the generated vibrations having the first frequency and a second focal point of the generated vibrations having the second frequency are aligned within the focused volume.

### BRIEF DESCRIPTION OF THE DRAWINGS

Various aspects and embodiments of the present disclosure are described hereinbelow with references to the drawings, wherein:

FIG. 1 is a perspective view of a system for selectively sealing small vessels using high intensity focused ultrasound in accordance with embodiments of the present disclosure;

FIG. 2 is a transverse cross-sectional view of an ultrasound transducer of the system of FIG. 1 in accordance with embodiments of the present disclosure;

FIG. 3 is a graphical illustration of a surgery using the system of FIG. 1 in accordance with embodiments of the present disclosure;

FIG. 4A is a block diagram of a system for selectively sealing small vessels using confocal and coaxial transducer elements in accordance with embodiments of the present disclosure;

FIG. 4B is a top view of the confocal and coaxial transducer elements of the system of FIG. 4A; and

FIG. 5 is a flow chart illustrating a method for selectively sealing small vessels in accordance with embodiments of the present disclosure.

### DETAILED DESCRIPTION

The present disclosure is directed to a system and method for selectively exposing and sealing a network of vessels in a liver and for performing liver resection by selectively sealing and resecting the vessel network. The system utilizes at least two transducers to generate ultrasound waves, which are to be transmitted through a matching layer, which may be sound transmission liquid and acoustic lens, to selectively break liver parenchyma. The ultrasound waves generated by one ultrasound transducer have insufficient energy to seal a small vessel. However, when the ultrasound waves generated by at least two ultrasound transducers are converged, combined, and superimposed at a focused volume, thermal and mechanical energy caused by the superimposed ultrasound waves have enough energy to seal the small vessels within the focused volume. Such thermal and mechanical energy, however, is not sufficient enough to seal a large vessel. In this way, small vessels are selectively sealed by the at least two ultrasound transducers.

With features described below, selective coagulation of a capillary network of a liver is realized. Complications after liver resection operations, such as heat injuries and bleeding of intra-operation or post-operation, are reduced. No puncture is needed for surgeries on the liver. Further, bile leakage is reduced after operations. Details of selective sealing of small vessels are described below with reference to figures.

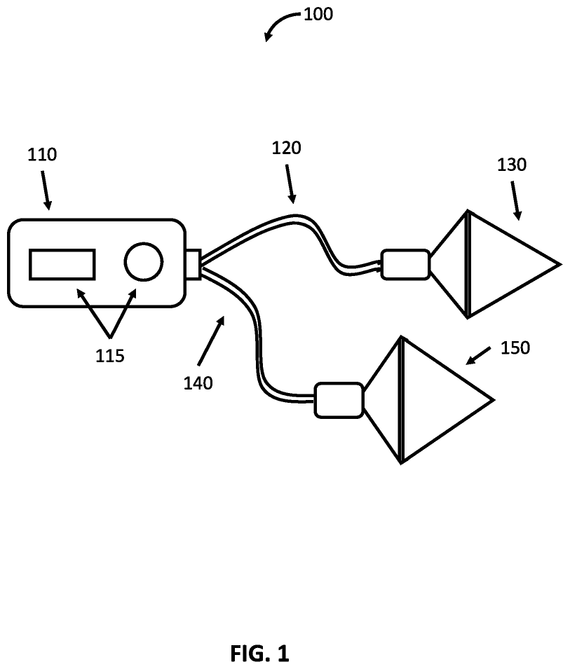

FIG. 1 shows a high intensity focused ultrasound (HIFU) system 100 for selectively sealing small vessels in a liver using HIFU. The HIFU system 100 includes a generator 110, a first transmission line 120, a first acoustic assembly 130, a second transmission line 140, and a second acoustic assembly 150. The HIFU system 100 controls the first acoustic assembly 130 and the second acoustic assembly 150 to focus ultrasound waves at a target tissue in a way similar to using a magnifying glass to focus sunlight. By focusing the ultrasound waves, the target within the focused volume is vibrated and heated up to cause cavitation and sealing.

In the path of ultrasound transmission, positive and negative pressures occur alternatively. When the acoustic energy is in a high level, meaning that the acoustic energy is higher than 0.1 watts/cm², microbubbles are formed in the tissue on the path of ultrasound transmission. Cavitation refers to such formation of microbubbles in the tissue. The microbubbles grow, deform, expand, and collapse in the process caused by the pressure changes. While the microbubbles are growing, deforming, and expanding, temperatures go high inside of the microbubbles.

When the microbubbles are collapsing, burst caused by the collapsing produces a shock wave to tissue near the collapsing and jets which can mechanically break the tissue surrounding the microbubbles. This mechanical and thermal effects caused by the shock wave and jets make vessels shrink in a short time, during which denaturization and coagulation of protein occurs. Walls of vessels undergo coagulation necrosis under the exposure of ultrasound energy in several seconds. In this way, small vessels, of which diameter is less than or equal to 0.2 millimeters (mm), can be sealed.

The generator 110 may be configured to generate electric power sufficient and suitable for causing the first acoustic assembly 130 to generate ultrasound energy. The generator 110 may include controllers 115 such as a knob to control parameters for the generator 110 or a touch screen to graphically control parameters for the generator 110. The controllers 115 of the generator 110 may also be a dip switch. The generator 110 may be activated by an operator. In an aspect, the generator 110 may be activated by a switch mechanically or electrically coupled with the first acoustic assembly 130 or the second acoustic assembly 150, so that, when the first acoustic assembly 130 or the second acoustic assembly 150 is activated, the generator 110 can be correspondingly activated.

In an aspect, the controllers 115 may include a phase controller. The phase controller may be configured to dynamically adjust relative phases of elements in an array of transducers of the first acoustic assembly 130 and the second acoustic assembly 150. Based on the adjusted phases, generated ultrasound waves by the first acoustic assembly 130 and the second acoustic assembly 150 may be steered to different locations or focused at a focal point. Further, the phase controller may be capable of correcting aberrations in the ultrasound beam due to tissue structures.

The generator 100 may include a processor (e.g., a central processing unit or a graphical processing unit) and a memory (e.g., a random-access memory, read-only memory, hard disk drive, solid state disk, a magnetic tape, etc.). The processor is configured to execute processor-executable instructions (e.g., modules, programs, batch files, etc.) stored in the memory. When the instructions are executed, the processor is configured to perform controls or operations of the generators, as described above and below.

In an aspect, each of the first acoustic assembly 130 and the second acoustic assembly 150 may have a lens which focuses the generated ultrasound waves at a focal point in a similar way as a magnifying glass focuses sunlight.

In still yet another aspect, each of the first acoustic assembly 130 and the second acoustic assembly 150 may have a spherically curved transducer so that generated ultrasound waves can be focused at a focal point, where the ultrasound waves converge.

The ultrasound energy generated by the generator 110 is transmitted to the first acoustic assembly 130 and the second acoustic assembly 150 via first transmission line 120 and the second transmission line 140, respectively. The electric power transmitted to the first acoustic assembly 130 via first transmission line 120 may be same or different from the electric power transmitted to the second acoustic assembly 150 via second transmission line 140. In a case when, the generator 110 may include at least two output ports so that the electric power transmitted to the first acoustic assembly 130 via first transmission line 120 may be different from the electric power transmitted to the second acoustic assembly 150. Each output port may output a predetermined amount of the electric power.

In case the generator 110 has one port, a power splitter may be used to split the electric power into two so that the first transmission line 120 and the second transmission line 140 may transmit the equally split electric power to the first acoustic assembly 130 and the second acoustic assembly 150, respectively.

The first transmission line 120 and the second transmission line 140 may be made of an electrically conductive material, such as copper, silver, gold, or any material suitable for transmission of the electric power. Topological shapes of the first transmission line 120 and the second transmission line 140 may affect efficiencies in transmission of the electric power. Thus, in order to adjust the amount of the transmitted electric power, shapes of the first transmission line 120 and the second transmission line 140 may be accordingly adjusted. For example, the first transmission line 120 and the second transmission line 140 may be maintained straight or may have one or more circular turns. Further, in order to prevent electromagnetic effects from each other, the first transmission line 120 and the second transmission line 140 may be coated or covered with an insulative material and shielded with a conducting material. Other preventative measures may be applied to the first transmission line 120 and the second transmission line 140 as readily appreciated by a person skilled in transmission lines for the HIFU system 100.

When receiving the electric power, the first acoustic assembly 130 and the second acoustic assembly 150 convert the electric power to ultrasound energy having a frequency. The frequency generated by the first acoustic assembly 130 and the frequency generated by the second acoustic assembly 150 may have the same fundamental frequency f₁. In an aspect, the frequencies generated by the first acoustic assembly 130 and the second acoustic assembly 150 may be equal to f₁. In another aspect, the frequency f₁ generated by the first acoustic assembly 130 may be less than the frequency f₂ generated by the second acoustic assembly 150, meaning that f₂=m*f₁, wherein m is an integer greater than or equal to two.

In an aspect, the generator 110 may activate one output port so that only one of the first acoustic assembly 130 and the second acoustic assembly 150 may receive the electric power. In this case, the ultrasound energy generated by the one may cause histotripsy within the focused volume. When two output ports are activated, the ultrasound energy generated by the first acoustic assembly 130 and the second acoustic assembly 150 may be superimposed or combined within the focused volume so that the small blood vessels may be selectively coagulated and sealed.

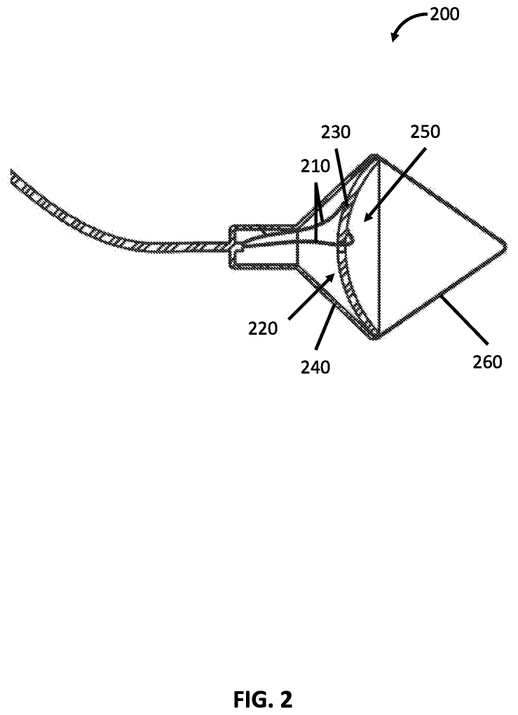

The first acoustic assembly 130 and the second acoustic assembly 150 may have the same or different structural dimension. In particular, one exemplary structure of an acoustic assembly 200 is shown in FIG. 2. The acoustic assembly 200 may be electrically coupled with the generator 110 via a transmission line and may be configured to generate ultrasound energy and focus the generated ultrasound energy to a focused volume. The focused volume may be about 1 cubic millimeter (mm³). By focusing the ultrasound energy in a small volume of tissue, the acoustic assembly 200 may be able to coagulate and seal small vessels located within the focused volume.

The acoustic assembly 200 may include an inner cable 210, a transducer 220, an electrode 230, a shell 240, a matching layer 250, and a lens 260. The inner cable 210 connects an electrical connection between the transmission line and the electrode 230 so that electric power is transmitted to the transducer 220. As shown in FIG. 2, the inner cable 210 may be connected to the front and back of the transducer 220 via the front and back electrodes 230. Then, the transducer 220 can convert the electric power to ultrasound energy. The material of the transducer 220 may be proteins, crystals (e.g. quartz), and/or ceramics. The transducer 220 may be enclosed by and fixed to the shell 240. The electric power may be transmitted to the transducer 220 by up to 500 watts, and in response, the transducer 220 may generate ultrasound waves having a high frequency (e.g., 250 KHz). The electrode 230 may have a thickness of about 0.1 mm so that the electrode 230 may have extremely low influence on the ultrasound vibrations generated by the transducer 220.

There is an acoustic impedance mismatch between the transducer 220 and water or tissue. For example, the acoustic impedance of the transducer 220, which may be made of ceramic, is about 35 mega Rayleigh (MRayl), while the acoustic impedance of water or tissue is about 1.5 MRayl. This difference in two materials in acoustic impedance causes sound reflection in the interface between the transducer 220 and tissue, and much of energy is wasted due to the reflection. Because of this, the acoustic assembly 200 may have the matching layer 250 between the transducer 220 and the tissue. The acoustic impedance of the matching layer 250 maybe set to be within a range of between about 10 and about 15 MRayl but is not limited to this range. The acoustic impedance of the matching layer 250 may be any acoustic impedance suitable for optimizing ultrasound wave transmission between the transducer 220 and the tissue. The matching layer 250 may be made of any material which can provide the optimal acoustic impedance in transmission of the ultrasound waves. For example, the matching layer 250 may be made of 1-3 butyl glycol.

The lens 260 may focus the ultrasound wave so that a focused volume in the tissue can be formed. As described above, the focused volume may be about 1 mm³. A sound transmission liquid may fill in the cavity between the transducer 220 and the lens 260. The lens 260 may be made of acrylonitrile butadiene styrene (ABS) plastic. The thickness of the lens 260 may be about 1 mm so that the loss of ultrasound energy is in a low range compared to the total ultrasound energy transmitted to the tissue. The lens 260 may be made of any materials having impact resistance, toughness, and heat resistance. Further, based on the field of view of the lens 260, the focused volume may be adjusted. The smaller the field of view, the smaller the focused volume may be.

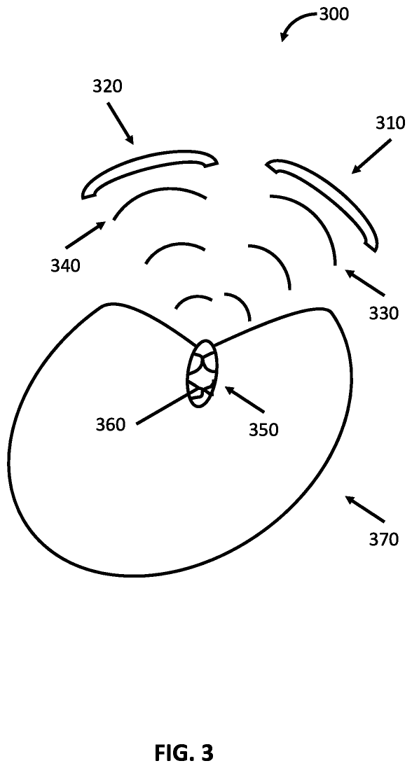

When the generator 110 is powered and provides electric power, the transducer 220 may generate ultrasound waves. As shown in FIG. 3, a first ultrasound transducer 310 generates ultrasound waves 330, and a second ultrasound transducer 320 generates ultrasound waves 340. Each of the ultrasound waves 330 and the ultrasound waves 340 has an insufficient thermal/mechanical energy to seal a small vessel in the tissue. Due to the changes in pressure of the ultrasound waves 330 and 340, a portion of the organ 370 may be retracted along the transmission directions of the ultrasound waves 330 and 340. When the ultrasound waves 330 and 340 are focused in the focused volume 350 of tissue in the organ 370, a network of small vessels 360 may be exposed within the focused volume and can be coagulated and sealed by the combined/superimposed ultrasound waves 330 and 340. The temperature within the focused volume may rise to 65° or 85° Celsius in seconds, destroying the small vessels by coagulative necrosis. The tissue surrounding the focused volume may be only minimally affected by the ultrasound waves 330 and 340 because the tissue surrounding the focused volume is exposed to only a small fraction of the ultrasound waves 330 and 340.

The size of the small vessels may be up to about 0.2 mm. Large vessels, of which diameters are larger than 0.2 mm, are not sealed even if the large vessels are located within the focused volume or volume, because the combined energy of the ultrasound wave 330 and 340 is not sufficient to seal the large vessels.

The first ultrasound transducer 310 may have a spherical shape with a first radius, r₁, and the second ultrasound transducer 320 may have another spherical shape with a second radius, r₂. The first radius may be equal to the second radius, namely r₁=r₂. In an aspect, the first radius r₁ and the second radius r₂ may be different from each other to adjust a location of the focused volume 350 or to adjust the amount of the combined or superimposed ultrasound energy.

The center of the first ultrasound transducer 310 may be the first focal point where the ultrasound waves generated by the first ultrasound transducer 310 converge, and the center of the second ultrasound transducer 320 may be the second focal point where the ultrasound waves generated by the second ultrasound transducer 320 converge. When the first and second focal points are positioned within the focused volume 350, the network of small vessels within the focused volume 350 are coagulated and sealed. In other words, when the first and second focal points are not positioned within the focused volume 350, the network of small vessels within the focused volume 350 is not sealed.

In other aspect, the first and second ultrasound transducers 310 and 320 may not have a spherical shape. Instead, the first ultrasound transducer 310 may generally have a first curvature and the second ultrasound transducer 320 may generally have a second curvature. The first curvature and the second curvature may be same or different. Based on the first and second curvatures, the first and second focal points are determined. When the first and second focal points are positioned within the focused volume 350, the network of small vessels within the focused volume 350 is coagulated and sealed. If not, the network of small vessels within the focused volume 350 is not sealed.

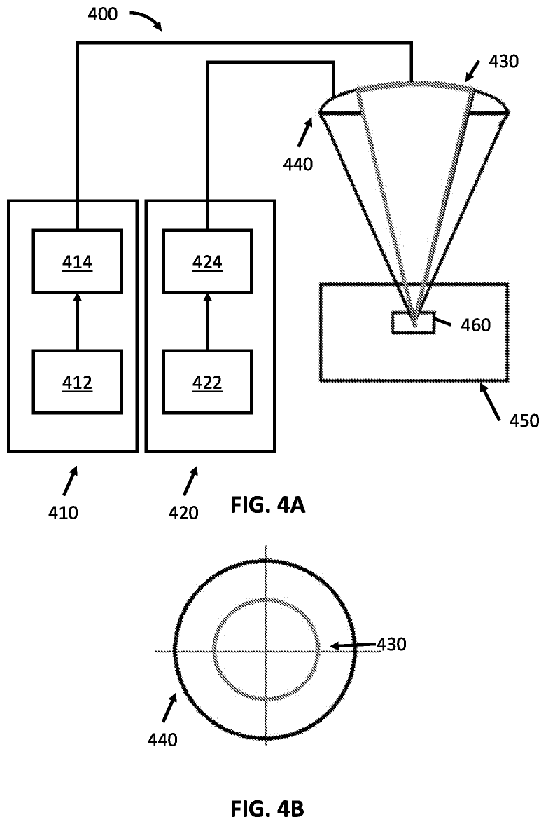

FIG. 4A illustrates a HIFU system 400 according to embodiments of the present disclosure. The HIFU system 400 includes a first generator 410 and a second generator 420. The first and second generators 410 and 420 include a function generator 412, 422, and a radio frequency (RF) amplifier 414, 424, respectively. The RF amplifiers 414 and 424 may adjust the output level of the electric power generated by the function generators 412 and 422, respectively. In an aspect, the function generator 412 and the RF amplifier 414 may provide electric power having a first frequency f₁, and the function generator 422 and the RF amplifier 424 may provide electric power having a second frequency f₂. The first and second frequencies f₁ and f₂ may have the same fundamental frequency.

The HIFU system 400 further includes a first transducer 430 and a second transducer 440. When receiving the electric power from the first and second generators 410 and 420, the first transducer 430 may generate ultrasound waves having the first frequency f₁, and the second transducer 430 may generate ultrasound waves having the second frequency f₂. In an aspect, the first and second transducers 430 and 440 may be made by cutting a spherical focused transducer into two coaxial and confocal transducer elements. Thus, the first and second transducers 430 and 440 may have the same fundamental frequency, meaning that f₂=m*f₁ where m is an integer greater than or equal to one.

In an aspect, the first and second transducers 430 and 440 may be aligned so that they have the same axis and the same focal point. FIG. 4B illustrates a top view of the first and second transducers 430 and 440, which have the same focus and the same axis, of the HIFU system 400 of FIG. 4A. In particular, the first transducer 430 is disposed inside of the second transducer 440. Further, combination of the first and second transducers 430 and 440 forms a portion of a spherical shape. In this way, the first and second transducers 430 and 440 can have the same focus and the same axis.

Thus, when the first and second transducers 430 and 440 generate ultrasound waves, it naturally follows that the generated ultrasound waves are focused at the focal point within a focused volume 460 of an organ 450 because the first and second transducers 430 and 440 are coaxial and confocal. Thus, in the HIFU system 400, there is no need to align the focal points of the first and second transducers 430 and 440 within the focused volume 460.

The focused volume 460 may be an ellipsoidal, spherical, or a cylindrical shape.

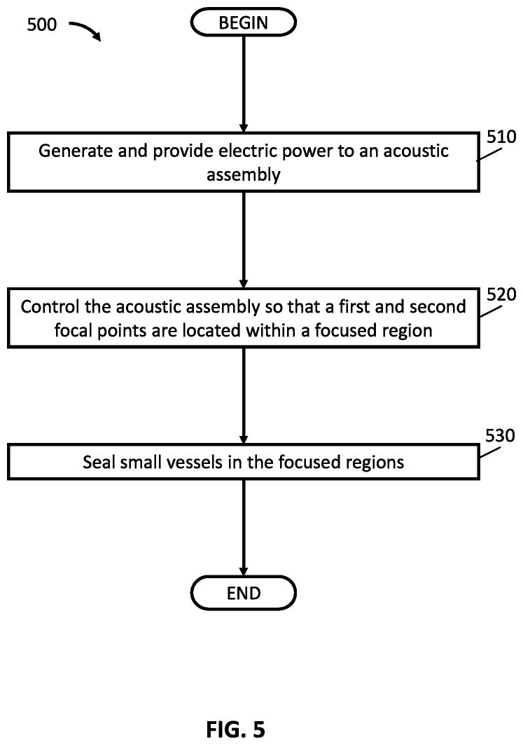

Returning to FIG. 5, a method 500 for selectively sealing small vessels is shown. In step 510, a generator of a HIFU system may generate and provide electric power to an acoustic assembly, which generates ultrasound vibrations. The acoustic assembly includes at least first and second ultrasound transducers.

In step 520, the acoustic assembly is controlled so that a first focal point of the first ultrasound transducer and a second focal point of the second ultrasound transducer are aligned within a focused volume. The focal point is a point where the generated ultrasound waves converge. The alignment of the focal points may be performed by the shape of the first and second ultrasound transducers. For example, the first and second ultrasound transducers may have a spherical shape. The first and second focal points may be the center of the spherical shape.

In an aspect, the acoustic assembly may include a lens, which causes the generated ultrasound waves to converge at the focal point. By adjusting the field of view of the lens, the focused volume may be enlarged or decreased. For example, the smaller the field of view is, the smaller the focused volume can be.

In another aspect, the HIFU system may include a phase controller, which dynamically adjusts relative phases of the elements in the array of the transducers. Based on the adjusted phases, generated ultrasound waves may be steered to different locations or focused at a focal point.

In step 530, when the first and second focal points are located within the focused volume, a network of small vessels in the focused volume is selectively sealed. The cross-sectional diameter of the small vessels may be less than or equal to 0.2 mm. If a vessel (i.e., large vessel) has a diameter larger than 0.2 mm, the large vessel is not sealed even when the large vessel is located within the focused volume. The ultrasound waves converged at the focal point do not have sufficient power to cause the large vessels to be sealed. In this way, the network of small vessels is selectively sealed with the acoustic assembly.

In an aspect, the power of the generator may adjust a level of the electric power so that large vessels located within the focused volume may be sealed.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A high intensity focused ultrasound (HIFU) system for selectively sealing a vessel network, the HIFU system comprising:
a generator configured to generate and supply electric power; and
an acoustic assembly configured to receive the supplied electric power, the acoustic assembly including:
a first transducer configured to generate vibrations having a first frequency; and
a second transducer configured to generate vibrations having a second frequency,
wherein, when a first focal point of the generated vibrations having the first frequency and a second focal point of the generated vibrations having the second frequency are aligned within a focused region, a group of vessels at the focused region are selectively sealed.

2. The HIFU system according to claim 1, wherein the first transducer has a first curvature and the second transducer has a second curvature.

3. The HIFU system according to claim 2, wherein the first curvature is equal to the second curvature.

4. The HIFU system according to claim 1, wherein the group of vessels is not sealed when the first focal point and the second focal point are not aligned within the focused region.

5. The HIFU system according to claim 1, wherein the second frequency is a harmonic frequency of the first frequency.

6. The HIFU system according to claim 1, wherein the group of vessels has a diameter less than or equal to 0.2 millimeters.

7. The HIFU system according to claim 1, wherein vessels, which have a diameter larger than 0.2 millimeters, are not sealed even when the first focal point and the second focal point are aligned within the focused region.

8. The HIFU system according to claim 1, further comprising a first lens coupled with the first transducer and a second lens coupled with the second transducer.

9. The HIFU system according to claim 8, wherein the first lens focuses the generated vibrations having the first frequency in the first focal point and the second lens focuses the generated vibrations having the second frequency in the second focal point.

10. The HIFU system according to claim 1, further comprising a phase controller configured to adjust relative phases of elements of the first transducer and of the second transducer.

11. The HIFU system according to claim 10, wherein the phase controller focuses the generated vibrations having the first frequency at the first focal point and focuses the generated vibrations having the second frequency at the second focal point.

12. A method for selectively sealing a vessel network with a high intensity focused ultrasound (HIFU) system including a first transducer and a second transducer, the method comprising:
providing electric power to the first transducer and the second transducer;
generating vibrations having a first frequency by the first transducer;
generating vibrations having a second frequency by the second transducer;
aligning a first focal point of the generated vibrations having the first frequency and a second focal point of the generated vibrations having the second frequency are aligned within a focused volume; and
selectively sealing a group of vessels located at a focused region, when a first focal point of the generated vibrations having the first frequency and a second focal point of the generated vibrations having the second frequency are aligned within the focused volume.

13. The method according to claim 12, wherein the vibration having the first frequency are generated by the first transducer having a first curvature, and
wherein the vibration having the second frequency are generated by the second transducer having a second curvature.

14. The method according to claim 12, wherein the electric power provided to the first transducer is equal to the electric power provided to the second transducer.

15. The method according to claim 12, wherein the electric power provided to the first transducer is different from the electric power provided to the second transducer.

16. The method according to claim 12, wherein the group of vessels is not sealed when the first focal point and the second focal point are not aligned within the focused volume.

17. The method according to claim 12, wherein the second frequency is a harmonic frequency of the first frequency.

18. The method according to claim 12, wherein the group of vessels is sealed when diameters of the group of vessels are less than or equal to 0.2 millimeters.

19. The method according to claim 12, wherein vessels, which have a diameter larger than 0.2 millimeters, are not sealed even when the first focal point and the second focal point are aligned within the focused volume.

20. A non-transitory computer readable medium including computer-executable instructions that, when executed by a computer, cause the computer to perform a method for selectively sealing a vessel network with a high intensity focused ultrasound (HIFU) system including a first transducer and a second transducer, the method comprising:
providing electric power to the first transducer and the second transducer;
generating vibrations having a first frequency by the first transducer; and
generating vibrations having a second frequency by the second transducer;
aligning a first focal point of the generated vibrations having the first frequency and a second focal point of the generated vibrations having the second frequency are aligned within a focused volume; and
selectively sealing a group of vessels located at a focused region, when a first focal point of the generated vibrations having the first frequency and a second focal point of the generated vibrations having the second frequency are aligned within the focused volume.
